(19) United States
(12) Patent Application Publication
(10) Pub. No.: US 2016/0121103 A1
(43) Pub. Date: May 5, 2016

(54) ELECTRICAL STIMULATION SYSTEM WITH ANCHORING STYLET AND METHODS OF MAKING AND USING

(71) Applicant: Boston Scientific Neuromodulation Corporation, Valencia, CA (US)

(72) Inventors: Geoffrey Abellana Villarta, Valencia, CA (US); Joshua Dale Howard, Winnetka, CA (US); G. Karl Steinke, Valencia, CA (US)

(21) Appl. No.: 14/929,133

(22) Filed: Oct. 30, 2015

Related U.S. Application Data
(60) Provisional application No. 62/074,468, filed on Nov. 3, 2014.

(51) Int. Cl. A61N 1/05

(52) U.S. Cl. CPC A61N 1/0534

(57) ABSTRACT

A stylet for use with an electrical stimulation lead includes a shaft having a proximal end portion and a distal end potion; a handle coupled to the proximal end portion; and at least one protuberance disposed along the proximal end portion of the shaft distal to the handle. The protuberances are configured and arranged for engaging a wall of a lumen of the electrical stimulation lead to hold the stylet in place within the lumen.

### CROSS-REFERENCE TO RELATED APPLICATIONS

This application claims the benefit under 35 U.S.C. §119(e) of U.S. Provisional Patent Application Ser. No. 62/074,468 filed Nov. 3, 2014, which is incorporated herein by reference.

### FIELD

The invention is directed to the area of electrical stimulation systems and leads and methods of making and using the systems and leads. The present invention is also directed to electrical stimulation leads with segmented electrodes formed from pre-electrodes with exterior depressions or apertures, as well as methods of making and using the segmented electrodes, leads, and electrical stimulation systems.

### BACKGROUND

Electrical stimulation can be useful for treating a variety of conditions. Deep brain stimulation can be useful for treating, for example, Parkinson's disease, dystonia, essential tremor, chronic pain. Huntington's disease, levodopa-induced dyskinesias and rigidity, bradykinesia, epilepsy and seizures, eating disorders, and mood disorders. Typically, a lead with a stimulating electrode at or near a tip of the lead provides the stimulation to target neurons in the brain. Magnetic resonance imaging (“MRI”) or computerized tomography (“CT”) scans can provide a starting point for determining where the stimulating electrode should be positioned to provide the desired stimulus to the target neurons.

After the lead is implanted into a patient's brain, electrical stimulus current can be delivered through selected electrodes on the lead to stimulate target neurons in the brain. Typically, the electrodes are formed into rings disposed on a distal portion of the lead. The stimulus current projects from the ring electrodes equally in every direction. Because of the ring shape of these electrodes, the stimulus current cannot be directed to one or more specific positions around the ring electrode (e.g., on one or more sides, or points, around the lead). Consequently, undirected stimulation may result in unwanted stimulation of neighboring neural tissue, potentially resulting in undesired side effects.

### BRIEF SUMMARY

One embodiment is a stylet configured and arranged for insertion into an electrical stimulation lead. The stylet includes a shaft having a proximal end portion and a distal end potion; a handle coupled to the proximal end portion; and at least one protuberance disposed along the proximal end portion of the shaft distal to the handle. The protuberances are configured and arranged for engaging a wall of a lumen of the electrical stimulation lead to hold the stylet in place within the lumen.

In at least some embodiments, the at least one protuberance is a plurality of protuberances. In at least some embodiments, the shaft and the at least one protuberance are formed of different materials. In at least some embodiments, the handle includes a plastic tube and a filler material, selected from silicone or polyurethane, disposed within the plastic tube. In at least some embodiments, the stylet includes an alignment feature disposed on the handle of the stylet.

Another embodiment is a kit for implantation of an electrical stimulation lead. The kit includes any of the stylets describe above; and an electrical stimulation lead having a lead body defining a central lumen for receiving the stylet, a proximal end portion, and a distal end portion. The lead also includes at least one electrode disposed along the distal end portion of the lead body, at least one terminal disposed along the proximal end portion of the lead body, and at least one conductor electrically coupling the at least one electrode to the at least one terminal.

In at least some embodiments, the central lumen has a diameter and the at least one protuberance has an outer diameter, where the outer diameter of the at least one protuberance is greater than or equal to the diameter of the central lumen of the lead. In at least some embodiments, the lead has an outer diameter and the handle has an outer diameter, where the outer diameter of the handle is equal to the diameter of the lead. In at least some embodiments, a durometer of the handle is within 10% of a durometer of the lead body.

In at least some embodiments, the lead and stylet are configured and arranged such that, when the protuberances are disposed in the central lumen of the lead, the protuberances and the central lumen form a compression fit. In at least some embodiments, the lead and stylet are configured and arranged such that, when the protuberances are disposed in the central lumen of the lead, the protuberances and the central lumen form a friction fit or an interlocking fit.

Yet another embodiment is a method of using any of the kits or stylets described above. The method includes inserting the shaft of the stylet into the central lumen of the lead; and inserting the at least one protuberance into the central lumen of the lead to anchor the stylet within the lead.

In at least some embodiments, the method also includes coupling a microdrive unit to the handle of the stylet and operating the microdrive unit on the handle of the stylet to advance, retract, or rotate the lead. In at least some embodiments, inserting the at least one protuberance into the central lumen of the lead includes forming a compression fit between the at least one protuberance and the central lumen of the lead. In at least some embodiments, inserting the at least one protuberance into the central lumen of the lead includes forming a friction fit or an interlocking fit between the at least one protuberance and the central lumen of the lead. In at least some embodiments, the method also includes aligning an alignment feature disposed on the handy of the stylet with an alignment feature disposed on the lead.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying, drawings, wherein:

FIG. 1A is a schematic side view of one embodiment of a device for electrical stimulation, according to the invention;

FIG. 1B is a schematic side view of a second embodiment of a device for electrical stimulation, according to the invention;

FIG. 2 is a schematic diagram of radial current steering along various electrode levels along the length of a lead, according to the invention;

FIG. 3A is a perspective view of an embodiment of a portion of a lead having a plurality of segmented electrodes, according to the invention;

FIG. 3B is a perspective view of a second embodiment of a portion of a lead having a plurality of segmented electrodes, according to the invention;

FIG. 3C is a perspective view of a third embodiment of a portion of a lead having a plurality of segmented electrodes, according to the invention;

FIG. 3D is a perspective view of a fourth embodiment of a portion of a lead having a plurality of segmented electrodes, according to the invention:

FIG. 3E is a perspective view of a fifth embodiment of a portion of a lead having a plurality of segmented electrodes, according to the invention;

FIG. 3F is a perspective view of a sixth embodiment of a portion of a lead having a plurality of segmented electrodes, according to the invention;

FIG. 3G is a perspective view of a seventh embodiment of a portion of a lead having a plurality of segmented electrodes, according to the invention;

FIG. 3H is a perspective view of an eighth embodiment of a portion of a lead having a plurality of segmented electrodes, according to the invention;

FIG. 4 is a schematic side view (and partial cross-sectional view) of one embodiment of a stylet, according to the invention: and

FIG. 5 is a schematic side view (and partial cross-sectional view) of the stylet of FIG. 4 inserted into a proximal end of a lead, according to the invention.

### DETAILED DESCRIPTION

The invention is directed to the area of electrical stimulation systems and leads and methods of making and using the systems and leads. The present invention is also directed to electrical stimulation leads with segmented electrodes formed from pre-electrodes with exterior depressions or apertures, as well as methods of making and using the segmented electrodes, leads, and electrical stimulation systems.

A lead for deep brain stimulation can include stimulation electrodes, recording electrodes, or a combination of both. At least some of the stimulation electrodes, recording electrodes, or both are provided in the form of segmented electrodes that extend only partially around the circumference of the lead. These segmented electrodes can be provided in sets of electrodes, with each set having electrodes radially distributed about the lead at a particular longitudinal position. For illustrative purposes, the leads are described herein relative to use for deep brain stimulation, but it will be understood that any of the leads can be used for applications other than deep brain stimulation, including spinal cord stimulation, peripheral nerve stimulation, or stimulation of other nerves and tissues.

Suitable implantable electrical stimulation systems include, but are not limited to, a least one lead with one or more electrodes disposed on a distal end of the lead and one or more terminals disposed on one or more proximal ends of the lead. Leads include, for example, percutaneous leads and paddle leads. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,175,710; 8,224,450; 8,271,094; 8,295,944; 8.364,278; 8,391,985; and 8,688,235; and U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated by reference. In the discussion below, a percutaneous lead will be exemplified, but it will be understood that the stylets described herein can also be used with paddle leads. In addition, implantation of the lead within the brain is described in detail below, but it will be understood that the leads and stylets described herein can also be used for implantation at other sites in the body including, but not limited to, the spinal cord or adjacent or within any other nerve, organ, or body tissue to be electrically stimulated.

In at least some embodiments, a practitioner may determine the position of the target neurons using, for example, recording electrode(s) or any other sensor as part of the lead or separate from the lead and then position the stimulation electrode(s) accordingly. In some embodiments, the same electrodes can be used for both recording and stimulation. In some embodiments. separate leads can be used; one with recording electrodes which identify target neurons, and a second lead with stimulation electrodes that replaces the first after target neuron identification. In some embodiments, the same lead can include both recording electrodes and stimulation electrodes or electrodes can be used for both recording and stimulation.

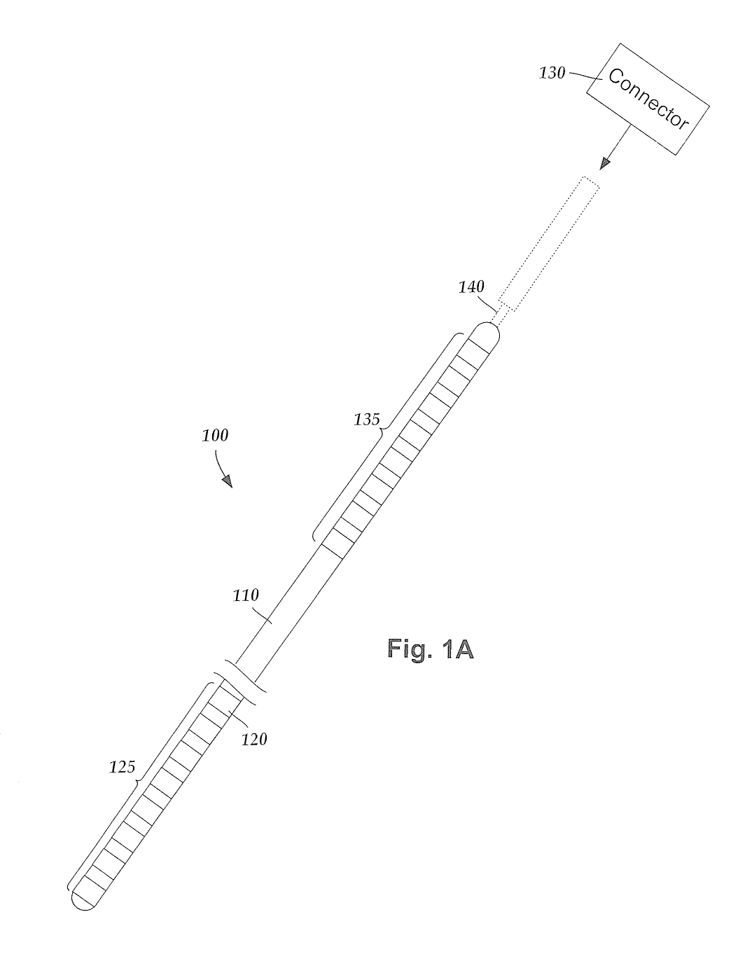

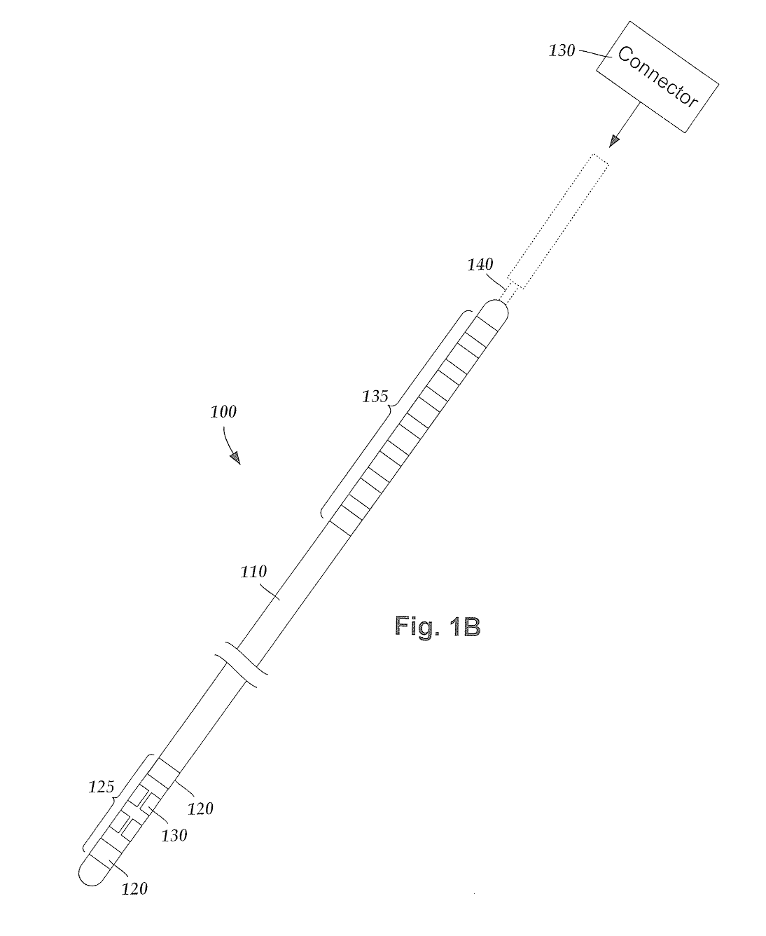

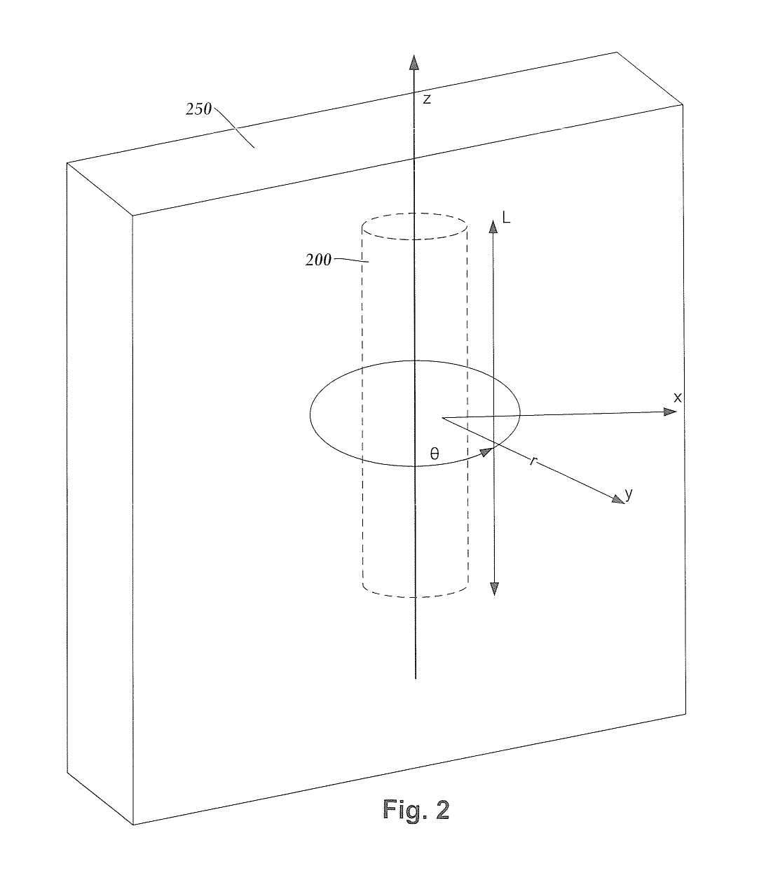

FIGS. 1A and 2B illustrates two embodiments of a device 100 for brain stimulation. The device includes a lead 110, a plurality of electrodes 125 disposed at least partially about a circumference of the lead 110, a plurality of terminals 135, a connector 132 for connection of the electrodes to a control unit, and a stylet 140 for assisting in insertion and positioning of the lead in the patient's brain. The connector 132 fits over a proximal end of the lead 110, preferably after removal of the stylet 140.

The control unit (not shown) is typically an implantable pulse generator that can be implanted into a patient's body, for example, below the patient's clavicle area or within the patient's buttocks or abdominal cavity. The pulse generator can have eight stimulation channels which may be independently programmable to control the magnitude of the current stimulus from each channel. In some cases the pulse generator can have more or fewer than eight stimulation channels (e.g., 4-, 6-, 16-, 32-, or more stimulation channels). The control unit can have one, two, three, four, or more connector ports, for receiving the plurality of terminals 135 at the proximal end of the lead 110.

In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 110 can be inserted into the cranium and brain tissue with the assistance of the stylet 140. The lead 110 can be guided to the target location within the brain using, for example, a stereotactic frame and a microdrive motor system. In some embodiments, the microdrive motor system can be fully or partially automatic. The microdrive motor system may be configured to perform one or more the following actions (alone or in combination): insert the lead 110, advance the lead 110, retract the lead 110, or rotate the lead 110.

In some embodiments, measurement devices coupled to the muscles or other tissues stimulated by the target neurons, or a unit responsive to the patient or clinician, can be coupled to the control unit or microdrive motor system. The measurement device, user, or clinician can indicate a response by the target muscles or other tissues to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in tremor frequency or amplitude in response to stimulation of neurons. Alternatively, the patient or clinician can observe the muscle and provide feedback.

The lead 110 for deep brain stimulation can include stimulation electrodes, recording electrodes, or both. In at least some embodiments, the lead 110 is rotatable so that the stimulation electrodes can be aligned with the target neurons after the neurons have been located using the recording electrodes.

Stimulation electrodes may be disposed on the circumference of the lead 110 to stimulate the target neurons. Stimulation electrodes may be ring-shaped so that current projects from each electrode equally in every direction from the position of the electrode along a length of the lead 110. In the embodiment of FIG. 1A, all of the electrodes are ring electrodes. In the embodiment of FIG. 1B, two of the electrodes are ring electrodes. Ring electrodes typically do not enable stimulus current to be directed from only a limited angular range around of the lead. Segmented electrodes, however, can be used to direct stimulus current to a selected angular range around the lead. When segmented electrodes are used in conjunction with an implantable pulse generator that delivers constant current stimulus, current steering can be achieved to more precisely deliver the stimulus to a position around an axis of the lead (i.e., radial positioning around the axis of the lead).

To achieve current steering, segmented electrodes can be utilized in addition to, or as an alternative to, ring electrodes. Though the following description discusses stimulation electrodes, it will be understood that all configurations of the stimulation electrodes discussed may be utilized in arranging recording electrodes as well.

The lead 100 includes a lead body 110, one or more optional ring electrodes 120, and a plurality of sets of segmented electrodes 130. The lead body 110 can be formed of a biocompatible, non-conducting material such as, for example, a polymeric material. Suitable polymeric materials include, but are not limited to, silicone, polyurethane, polyurea, polyurethane-urea, polyethylene, or the like. Once implanted in the body, the lead 100 may be in contact with body tissue for extended periods of time. In at least some embodiments, the lead 100 has a cross-sectional diameter of no more than 1.5 mm and may be in the range of 0.5 to 1.5 mm. In at least some embodiments, the lead 100 has a length of at least 10 cm and the length of the lead 100 may be in the range of 10 to 70 cm.

The electrodes can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes are made of a material that is biocompatible and does not substantially corrode under expected operating conditions in the operating environment for the expected duration of use.

Each of the electrodes can either be used or unused (OFF). When the electrode is used, the electrode can be used as an anode or cathode and carry anodic or cathodic current. In some instances, an electrode might be an anode for a period of time and a cathode for a period of time.

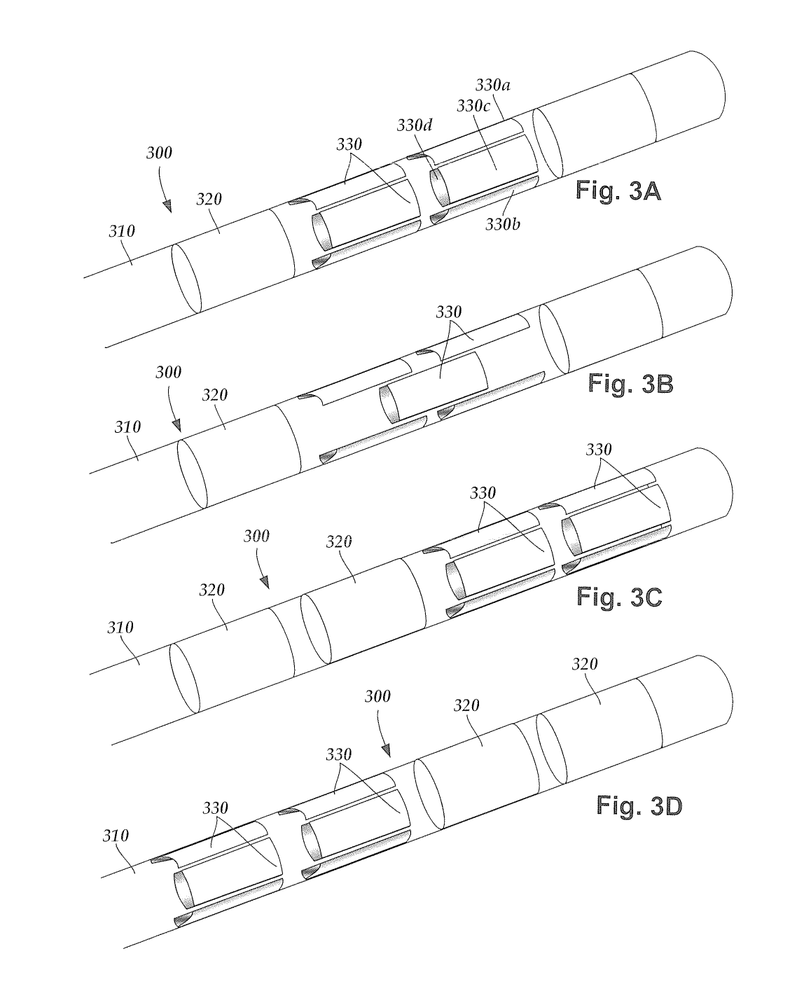

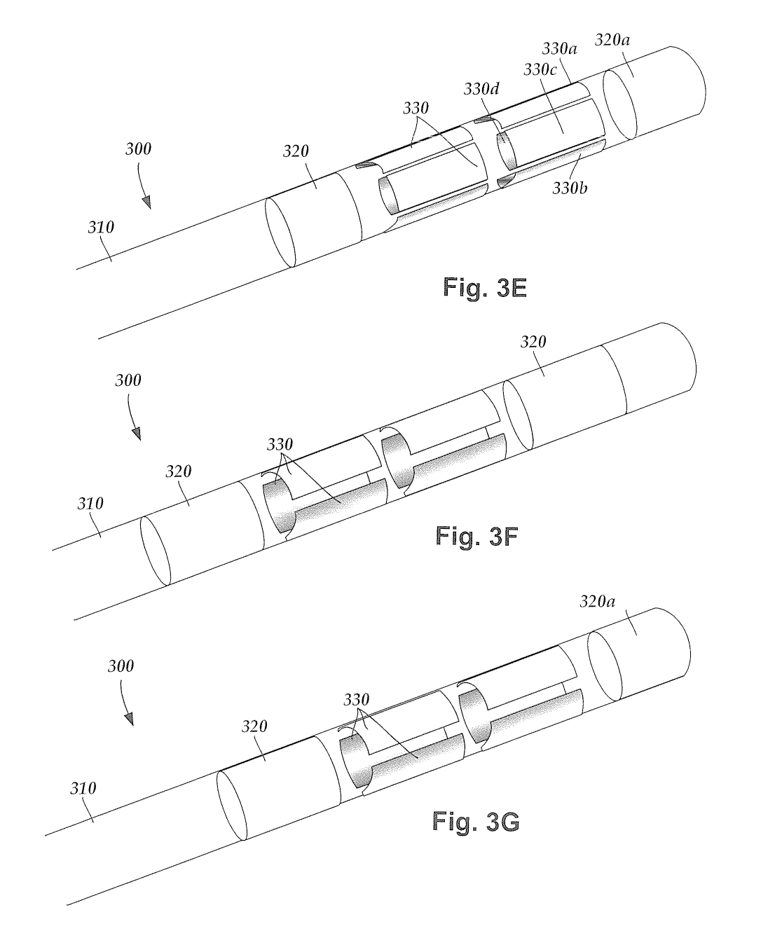

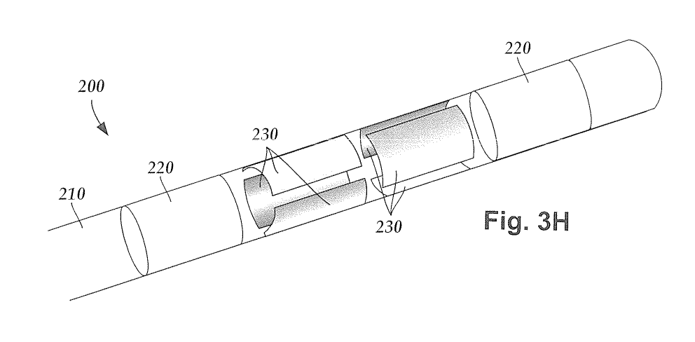

Stimulation electrodes in the form of ring electrodes 120 can be disposed on any part of the lead body 110, usually near a distal end of the lead 100. In FIG. 1A, the lead 100 includes eight ring electrodes. In FIG. 1B, the lead 100 includes two ring electrodes 120. Any number of ring electrodes 120 can be disposed along the length of the lead body 110 including, for example, one, two three, four, five, six, seven, eight, nine, ten, eleven, twelve, thirteen, fourteen, fifteen, sixteen or more ring electrodes 120. It will be understood that any number of ring electrodes can be disposed along the length of the lead body 110. In some embodiments, the ring electrodes 120 are substantially cylindrical and wrap around the entire circumference of the lead body 110. In some embodiments, the outer diameters of the ring electrodes 120 are substantially equal to the outer diameter of the lead body 110. The length of the ring electrodes 120 may vary according to the desired treatment and the location of the target neurons. In some embodiments the length of the ring electrodes 120 are less than or equal to the diameters of the ring electrodes 120. In other embodiments, the lengths of the ring electrodes 120 are greater than the diameters of the ring electrodes 120. The distal-most ring electrode 120 may be a tip electrode (see, e.g., tip electrode 320a of FIG. 3E) which covers most, or all, of the distal tip of the lead.

Deep brain stimulation leads may include one or more sets of segmented electrodes. Segmented electrodes may provide for superior current steering than ring electrodes because target structures in deep brain stimulation are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This 115 provides precise three-dimensional targeting and delivery of the current stimulus to neural target tissue, while potentially avoiding stimulation of other tissue. Examples of leads with segmented electrodes include U.S. Patent Application Publication Nos. 2010/0268298; 2011/0005069; 2011/0130803; 2011/0130816; 2011/0130817; 2011/0130818; 2011/0078900; 2011/0238129; 2012/0016378; 2012/0046710; 2012/0071949; 2012/0165911; 2012/197375; 2012/0203316; 2012/0703370; 2012/0703321, all of which are incorporated herein by reference.

The lead 100 is shown having a plurality of segmented electrodes 130. Any number of segmented electrodes 130 may be disposed on the lead body 110 including, for example, one, two three, four, five, six, seven, eight, nine, ten, eleven, twelve, thirteen, fourteen, fifteen, sixteen or more segmented electrodes 130. It will be understood that any number of segmented electrodes 130 may be disposed along the length of the lead body 110. A segmented electrode 130 typically extends only 75%, 67%, 60%, 50%, 40%, 33%, 25%, 20%, 17%, 15%, or less around the circumference of the lead.

The segmented electrodes 130 may be grouped into sets of segmented electrodes, where each set is disposed around a circumference of the lead 100 at a particular longitudinal portion of the lead 100. The lead 100 may have any number segmented electrodes 130 in a given set of segmented electrodes. The lead 100 may have one, two, three, four, five, six, seven, eight, or more segmented electrodes 130 in a given set. In at least some embodiments, each set of segmented electrodes 130 of the lead 100 contains the same number of segmented electrodes 130. The segmented electrodes 130 disposed on the lead 100 may include a different number of electrodes than at least one other set of segmented electrodes 130 disposed on the lead 100.

The segmented electrodes 130 may vary in size and shape. In some embodiments, the segmented electrodes 130 are all of the same size, shape, diameter, width or area or any combination thereof. In some embodiments, the segmented electrodes 130 of each circumferential set (or even all segmented electrodes disposed on the lead 100) may be identical in size and shape.

Each set of segmented electrodes 130 may be disposed around the circumference of the lead body 110 to form a substantially cylindrical shape around the lead body 110. The spacing between individual electrodes of a given set of the segmented electrodes may be the same, or different from, the spacing between individual electrodes of another set of segmented electrodes on the lead 100. In at least some embodiments, equal spaces, gaps or cutouts are disposed between each segmented electrode 130 around the circumference of the lead body 110. In other embodiments, the spaces, gaps or cutouts between the segmented electrodes 130 may differ in size or shape. In other embodiments, the spaces, gaps, or cutouts between segmented electrodes 130 may be uniform for a particular set of the segmented electrodes 130, or for all sets of the segmented electrodes 130. The sets of segmented electrodes 130 may be positioned in irregular or regular intervals along a length the lead body 110.

Conductor wires that attach to the ring electrodes 120 or segmented electrodes 130 extend along the lead body 110. These conductor wires may extend through the material of the lead 100 or along one or more lumens defined by the lead 100, or both. The conductor wires couple the electrodes 120, 130 to the terminals 135.

When the lead 100 includes both ring electrodes 120 and segmented electrodes 130, the ring electrodes 120 and the segmented electrodes 130 may be arranged in any suitable configuration. For example, when the lead 100 includes two ring electrodes 120 and two sets of segmented electrodes 130, the ring electrodes 120 can flank the two sets of segmented electrodes 130 (see e.g, FIGS. 1, 3A, and 3E-3H—ring electrodes 320 and segmented electrode 330). Alternately, the two sets of ring electrodes 120 can be disposed proximal to the two sets of segmented electrodes 130 (see e.g., FIG. 3C—ring electrodes 320 and segmented electrode 330), or the two sets of ring electrodes 120 can be disposed distal to the two sets of segmented electrodes 130 (see e.g., FIG. 3D—ring electrodes 320 and segmented electrode 330). One of the ring electrodes can be a tip electrode (see, tip electrode 320a of FIGS. 3E and 3G). It will be understood that other configurations are possible as well (e.g., alternating ring and segmented electrodes, or the like).

By varying the location of the segmented electrodes 130, different coverage of the target neurons may be selected. For example, the electrode arrangement of FIG. 3C may be useful if the physician anticipates that the neural target will be closer to a distal tip of the lead body 110, while the electrode arrangement of FIG. 3D may be useful if the physician anticipates that the neural target will be closer to a proximal end of the lead body 110.

Any combination of ring electrodes 120 and segmented electrodes 130 may be disposed on the lead 100. For example, the lead may include a first ring electrode 120, two sets of segmented electrodes; each set formed of four segmented electrodes 130, and a final ring electrode 120 at the end of the lead. This configuration may simply be referred to as a 1-4-4-1 configuration (FIGS. 3A and 3E—ring electrodes 320 and segmented electrode 330). It may be useful to refer to the electrodes with this shorthand notation. Thus, the embodiment of FIG. 3C may be referred to as a 1-1-4-4 configuration, while the embodiment of FIG. 3D may be referred to as a 4-4-1-1 configuration. The embodiments of FIGS. 3F, 3G, and 3H can be referred to as a 1-3-3-1 configuration. Other electrode configurations include, for example, a 2-2-2-2 configuration, where four sets of segmented electrodes are disposed on the lead, and a 4-4 configuration, where two sets of segmented electrodes, each having four segmented electrodes 130 are disposed on the lead. The 1-3-3-1 electrode configuration of FIGS. 3F, 3G, and 3H has two sets of segmented electrodes, each set containing three electrodes disposed around the circumference of the lead, flanked by two ring electrodes (FIGS. 3F and 3H) or a ring electrode and a tip electrode (FIG. 3G). In some embodiments, the lead includes 16 electrodes. Possible configurations for a 16-electrode lead include, but are not limited to 4-4-4-4; 8-8; 3-3-3-3-3-1 (and all rearrangements of this configuration); and 2-2-2-2-2-2-2-2.

FIG. 2 is a schematic diagram to illustrate radial current steering along various electrode levels along the length of the lead 200. While conventional lead configurations with ring electrodes are only able to steer current along the length of the lead (the z-axis), the segmented electrode configuration is capable of steering current in the x-axis, y-axis as well as the z-axis. Thus, the centroid of stimulation may be steered in any direction in the three-dimensional space surrounding the lead 200. In some embodiments, the radial distance, r, and the angle θ around the circumference of the lead 200 may be dictated by the percentage of anodic current (recognizing that stimulation predominantly occurs near the cathode, although strong anodes may cause stimulation as well) introduced to each electrode. In at least some embodiments, the configuration of anodes and cathodes along the segmented electrodes allows the centroid of stimulation to be shifted to a variety of different locations along the lead 200.

As can be appreciated from FIG. 2, the centroid of stimulation can be shifted at each level along the length of the lead 200. The use of multiple sets of segmented electrodes at different levels along the length of the lead allows for three-dimensional current steering. In some embodiments, the sets of segmented electrodes are shifted collectively (i.e., the centroid of simulation is similar at each level along the length of the lead). In at least some other embodiments, each set of segmented electrodes is controlled independently. Each set of segmented electrodes may contain two, three, four, five, six, seven, eight or more segmented electrodes. It will be understood that different stimulation profiles may be produced by varying the number of segmented electrodes at each level. For example, when each set of segmented electrodes includes only two segmented electrodes, uniformly distributed gaps (inability to stimulate selectively) may be formed in the stimulation profile. In some embodiments, at least three segmented electrodes 230 in a set are utilized to allow for true360° selectivity.

As previously indicated, the foregoing configurations may also be used while utilizing recording electrodes. In some embodiments, measurement devices coupled to the muscles or other tissues stimulated by the target neurons or a unit responsive to the patient or clinician can be coupled to the control unit or microdrive motor system. The measurement device, user, or clinician can indicate a response by the target muscles or other tissues to the stimulation or recording electrodes to further identify the target neurons and facilitate positioning of the stimulation electrodes. For example, if the target neurons are directed to a muscle experiencing tremors, a measurement device can be used to observe the muscle and indicate changes in tremor frequency or amplitude in response to stimulation of neurons. Alternatively, the patient or clinician may observe the muscle and provide feedback.

The reliability and durability of the lead will depend heavily on the design and method of manufacture. Fabrication techniques discussed below provide methods that can produce manufacturable and reliable leads.

Returning to FIG. 1B, when the lead 100 includes a plurality of sets of segmented electrodes 130, it may be desirable to form the lead 100 such that corresponding electrodes of different sets of segmented electrodes 130 are radially aligned with one another along the length of the lead 100 (see e.g., the segmented electrodes 130 shown in FIG. 1B). Radial alignment between corresponding electrodes of different sets of segmented electrodes 130 along the length of the lead 100 may reduce uncertainty as to the location or orientation between corresponding segmented electrodes of different sets of segmented electrodes. Accordingly, it may be beneficial to form electrode arrays such that corresponding electrodes of different sets of segmented electrodes along the length of the lead 100 are radially aligned with one another and do not radially shift in relation to one another during manufacturing of the lead 100.

In other embodiments, individual electrodes in the two sets of segmented electrodes 130 are staggered (see, FIG. 3B) relative to one another along the length of the lead body 110. In some cases, the staggered positioning of corresponding, electrodes of different sets of segmented electrodes along the length of the lead 100 may be designed for a specific application.

Segmented electrodes can be used to tailor the stimulation region so that, instead of stimulating tissue around the circumference of the lead as would be achieved using a ring electrode, the stimulation region can be directionally targeted. In some instances, it is desirable to target a parallelepiped (or slab) region 250 that contains the electrodes of the lead 200, as illustrated in FIG. 2. One arrangement for directing a stimulation field into a parallelepiped region uses segmented electrodes disposed on opposite sides of a lead.

FIGS. 3A-3H illustrate leads 300 with segmented electrodes 330, optional ring electrodes 320 or tip electrodes 320a, and a lead body 310. The sets of segmented electrodes 330 each include either two (FIG. 3B), three (FIGS. 3E-3H), or four (FIGS. 3A, 3C, and 3D) or any other number of segmented electrodes including, for example, three, five, six, or more. The sets of segmented electrodes 330 can be aligned with each other (FIGS. 3A-3G) or staggered (FIG. 3H)

Any other suitable arrangements of segmented electrodes can be used. As an example, arrangements in which segmented electrodes are arranged helically with respect to each other. One embodiment includes a double helix.

Conventionally, a stylet is inserted into a lead during implantation of the lead to provide stiffness to the lead. The stylet typically slides freely within the lead and includes a handle to facilitate insertion into the lead and removal from the lead. The handle may also provide a stop for the insertion of the stylet into the lead as the handle reaches the end of the lead.

In contrast to a conventional stylet, the stylet described herein in arranged to anchor to the lead when operationally inserted into the lead so that the stylet can be used to insert, advance, retract, or rotate the lead by manipulation of the stylet alone. For example, the stylet can include one or more protuberances that interact with the wall of the stylet lumen of the lead to fix the position of the stylet relative to the lead so that the stylet can be used to insert, advance, retract, or rotate the lead by manipulation of the stylet alone. This may be advantageous, particularly in circumstances in which it is desired to avoid damage to the lead or when the lead is short. For example, using this stylet, a microdrive device can be coupled to the stylet and used to insert, advance, retract, or rotate a lead in contrast to conventional methods which directly couple the microdrive device to the lead. Such direct coupling may not be possible if, for example, the lead is short.

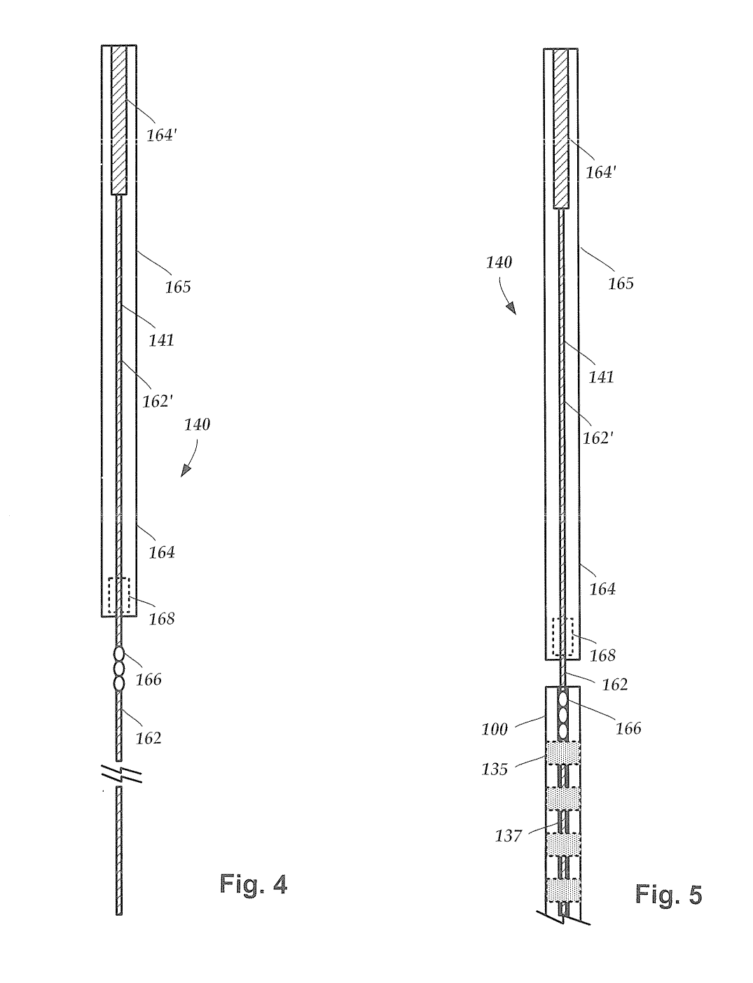

FIG. 4 illustrates one example of a stylet 140. The stylet includes a shaft 162, a handle 164, and one or more protuberances 166 disposed distal to the handle. In the illustrated embodiment, the stylet 140 has been modified from a commercially available stylet 141 (cross-hatched) with a shaft 162′ and a handle 164′ by adding an additional handle portion 165. In other embodiments, the handle 164 can simply be a proximal end of the shaft 162 or can be attached to the shaft 162 by a practitioner or other person prior to, or during use, of the stylet. It will be understood, however, that the stylet does not need to be modified from a commercially available stylet. The stylet 140 may also include an alignment feature 168 that may be aligned with a corresponding alignment feature on the lead which indicates the relative orientation of the segmented electrodes on the lead (see, for example, FIG. 1B). The alignment feature 168 can be, for example, at least one marker, marking, character, symbol, stripe, or any combination thereof.

FIG. 5 illustrates the stylet 140 inserted into the proximal end of a lead 100. The lead includes terminals 135 and a central lumen 137 for receiving the stylet 140. The one or more protuberances 166 have an outer diameter selected to be equal to or slightly larger than a diameter of the central lumen 137 so that the protuberances contact the wall of the central lumen and resist movement of the stylet 140 relative to the lead 100 absent application of a force exceeding a threshold level. In this manner, the interaction between the protuberances 166 and the wall of the central lumen 137 maintain the stylet 140 and lead 100 in a same relative position so that the stylet can be used to insert, advance, retract, or rotate the lead. The protuberances 166 and the wall of the central lumen 137 may form a frictional fit or the protuberances and wall of the central lumen may form a compression fit with the protuberances or wall (or both) compressing the other element. In some embodiments, the wall of the central lumen 137 can include features, such as protuberances or depressions or any combination thereof that interact with the protuberances 166 on the stylet 140 to form a friction fit, compression fit, interlocking fit, or any combination thereof.

The shaft 162 of the stylet 140 can be made of any suitable material including, but not limited to tungsten, stainless steel, other metals, rigid plastics, or any combination thereof. The shaft 162 fits within the central lumen 137 of the lead 100 to stiffen the lead during implantation and positioning of the lead within patient tissue. The shaft 162 (except at the one or more protuberances 166) has an outer diameter that is less than the diameter of the central lumen 137 of the lead 100 into which the stylet 140 is to be inserted. The length of the shaft 162 can be the same or exceed the length of the lead or the length of the shaft can be less than the length of the lead.

The handle 164 of the stylet 140 can be made of the same or different materials. In some embodiments, the handle, or a portion of the handle, may be made of a less rigid material than the shaft. Rigidity of the handle is less important than the shaft because the handle remains outside the lead. In at least some embodiments, the outer diameter of the handle 164 is larger than the outer diameter of the central lumen 137 of the lead 100.

In some embodiments, the handle 164 may be made of one or more materials that provide the handle with a durometer (a measure of hardness/softness of the material) that is the same as, or within 10% or 5% of, the durometer of the lead 100. Such an arrangement may be particularly useful if the microdrive unit is coupled to the handle 164 of the stylet 140 for insertion, advancement, retraction, or rotation of the lead. In some embodiments, the outer diameter of the handle 164 can be the same as, or within 10% or 5% of, the outer diameter of the lead 100. This may also facilitate coupling of the microdrive unit to the handle to insert, advance, retract, or rotate the lead.

The one or more protuberances 166 can be made of the same material as the shaft 162 and integral with the shaft. Alternatively, the one or more protuberances 166 can be made of a different material than the shaft 162 and may be formed on the shaft during or after manufacture of the stylet. For example, the one or more protuberances 166 can be added to the shaft 162 of an existing, or commercially available, stylet 140. The one or more protuberances can be made of metal; glue or epoxy; silicone, polyurethane, or other elastic materials; or the like. Preferably, the one or more protuberances 166 have an outer diameter that is equal to or slightly greater than (for example, up to 10% greater than) the diameter of the central lumen 137 of the lead 100 into which the stylet 140 is inserted. In at least some embodiments, the lead 100 is relatively soft and so the material of the lead forming the central lumen 137 can expand to admit the protuberances 166 and provide a compression effect between the protuberances and the wall of the central lumen of the lead. In some embodiments, the protuberances 166 can be relatively soft and be compressed by the wall of the central lumen of the lead.

The stylet 140 can have any number of protuberances 166 including one, two, three, four, five, or more protuberances. In at least some embodiments, the one or more protuberances 166 are disposed on the shaft 162 of the stylet 140 near the handle 166 (for example, within 0.5 to 5 cm of the handle.) In at least some embodiments, the stylet 140 is arranged so that the one or more protuberances 166 are intended to be inserted no more than 1 to 5 cm into the outer lumen 137 of the lead 100. A practitioner, however, may decide to insert the protuberances 166 further into the lead 100.

In at least some embodiments, the stylet 140 is formed from an existing, or commercially available, stylet by adding the protuberances 166 to the existing, or commercially available, stylet. In some embodiments, the handle 164 is formed from the handle 164′ of the existing, or commercially available, stylet by placing a plastic or metal tube 165 over the handle 164′ and portion of the shaft 162′ of the existing, or commercially available, stylet. In some embodiments, particularly if the tube 165 is made of plastic, the interior of the tube 165 may be filled with silicone, polyurethane, or other polymeric material. This can be used to give the handle 164 of the stylet 140 a durometer that is the same or close to the same (for example, within 10% or 5%) of the durometer of the lead 100. Such an arrangement may be particularly useful if the microdrive unit is coupled to the handle 164 of the stylet 140 for insertion, advancement, retraction, or rotation of the lead.

The optional alignment feature 168 can be, for example, at least one colored or metallic marker, marking, character, symbol, stripe, or any combination thereof and can be aligned with one or more segmented electrodes, if provided, of the lead 100. Alternatively, the alignment feature may be provided to give a practitioner a visual indication of twisting of the lead during implantation or rotation.

The above specification, examples, and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A stylet configured and arranged for insertion into an electrical stimulation lead, the stylet comprising:
a shaft comprising a proximal end portion and a distal end potion;
a handle coupled to the proximal end portion; and
at least one protuberance disposed along the proximal end portion of the shaft distal to the handle, wherein the protuberances are configured and arranged for engaging a wall of a lumen of the electrical stimulation lead to hold the stylet in place within the lumen.

2. The stylet of claim 1, wherein the at least one protuberance is a plurality of protuberances.

3. The stylet of claim 1, wherein the shaft and the at least one protuberance are formed of different materials.

4. The stylet of claim 1, wherein the handle comprises a plastic tube and a filler material, selected from silicone or polyurethane, disposed within the plastic tube.

5. The stylet of claim 1, further comprising an alignment feature disposed on the handle of the stylet.

6. A kit for implantation of an electrical stimulation lead, the kit comprising:
the stylet of claim 1; and
an electrical stimulation lead comprising a lead body defining a central lumen for receiving the stylet, a proximal end portion, and a distal end portion, the lead further comprising at least one electrode disposed along the distal end portion of the lead body, at least one terminal disposed along the proximal end portion of the lead body, and at least one conductor electrically coupling the at least one electrode to the at least one terminal.

7. The kit of claim 6, wherein the central lumen comprises a diameter and the at least one protuberance comprises an outer diameter, wherein the outer diameter of the at least one protuberance is greater than or equal to the diameter of the central lumen of the lead.

8. The kit of claim 6, wherein the lead comprises an outer diameter and the handle comprises an outer diameter, wherein the outer diameter of the handle is equal to the diameter of the lead.

9. The kit of claim 6, wherein a durometer of the handle is within 10% of a durometer of the lead body.

10. The kit of claim 6, wherein the lead and stylet are configured and arranged such that, when the protuberances are disposed in the central lumen of the lead, the protuberances and the central lumen form a compression fit.

11. The kit of claim 6, wherein the lead and stylet are configured and arranged such that, when the protuberances are disposed in the central lumen of the lead, the protuberances and the central lumen form a friction fit or an interlocking fit.

12. The kit of claim 6, wherein the at least one protuberance is a plurality of protuberances.

13. The kit of claim 6, wherein the shaft and the at least one protuberance are formed of different materials.

14. The kit of claim 6, wherein the handle comprises a plastic tube and a filler material, selected from silicone or polyurethane, disposed within the plastic tube.

15. The kit of claim 6, further comprising an alignment feature disposed on the handle of the stylet.

16. A method of using the kit of claim 6, the method comprising:
inserting the shaft of the stylet into the central lumen of the lead: and
inserting the at least one protuberance into the central lumen of the lead to anchor the stylet within the lead.

17. The method of claim 16, further comprising coupling a microdrive unit to the handle of the stylet and operating the microdrive unit on the handle of the stylet to advance, retract, or rotate the lead.

18. The method of claim 16, wherein inserting the at least one protuberance into the central lumen of the lead comprising forming a compression fit between the at least one protuberance and the central lumen of the lead.

19. The method of claim 16, wherein inserting the at least one protuberance into the central lumen of the lead comprising forming a friction fit or an interlocking fit between the at least one protuberance and the central lumen of the lead.

20. The method of claim 16, further comprising aligning an alignment feature disposed on the handle of the stylet with an alignment feature disposed on the lead.
